(19) United States
(12) Patent Application Publication
(10) Pub. No.: US 2018/0064530 A1
(43) Pub. Date: Mar. 8, 2018

(54) BREAST IMPLANTS

(71) Applicant: IMPLITE LTD., Petah Tikva (IL)

(72) Inventor: Ami GLICKSMAN, Petah Tikva (IL)

(21) Appl. No.: 15/551,330

(22) PCT Filed: Feb. 16, 2016
(86) PCT No.: PCT/IL2016/050178

§ 371 Date: Aug. 16, 2017

Related U.S. Application Data
(60) Provisional application No. 62/116,940, filed on Feb. 17, 2015.

(51) Int. Cl. A61F 2/12

(52) U.S. Cl. CPC A61F 2/12; A61F 2/0059; A61F 2250/0058

(57) ABSTRACT

There is provided herein a breast implant comprising: an internal skeleton element comprising a base configured to face a subject's chest wall when implanted, a dome configured to face a subject's nipple-areola complex when implanted, and a projecting structure extending between the base and the dome; and a shell enclosing the internal skeleton element and filled with a filler substance. The breast implant is configured to be inserted into a sub-glandular plane, a sub-muscular plane or a subcutaneous plane of a subject's breast, to affect the projection and volume of the breast.

### FIELD OF THE INVENTION

The present invention relates to breast implants.

### BACKGROUND

Breast implants have been in use since the early 1960's for reconstructing, improving contour or augmenting the female breast. The most common breast implants generally include a flexible elastomeric shell or envelope, typically made of silicone, which is filled with a soft gel, mainly silicone gel, a saline solution or a combination of both.

Current breast implants need to be replaced periodically. For example, silicone gel-filled implants need to be replaced every ten (10) years as indicated by the FDA or earlier if spontaneous rupture of the implant has been detected. Saline-filled implants also need to be replaced because of leakage and deflation of the implants. Current breast implants, such as the silicone gel-filled implants and the saline-filled implants, are relatively heavy, with specific gravity ranging between 1.0-1.1. The combination of implant's weight and aging skin with diminished skin elasticity results in breast ptosis and breast irregularities, sometimes requiring breast lifting and reshaping procedures.

There still remains a need for improved breast implants.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The present invention provides, according to some aspects, three dimensional breast implants comprising an internal skeleton element composed of a base, a dome and an elongated projecting structure connecting the base and dome, and further comprising a shell enclosing the skeleton element and filled with a filler substance. In some embodiments, the breast implants further comprise one or more reinforcing structures between the dome and projecting structure, between the base and projecting structure, or between the dome, base and projecting structure. In additional embodiments, the breast implants further comprise anchoring structures connecting the shell to the internal skeleton element, thus maintaining these components in a fixed position with respect to each other. The breast implants disclosed herein provide improved support in terms of maintaining shape and projection of an implanted breast, yet are light-weight compared to known implants.

The breast implants disclosed herein are useful both as primary implants, to augment a breast and obtain increased breast projection, and also as secondary implants, to replace older implants that need to be removed. According to some embodiments, there are provided herein breast implants that address the need for periodic replacement of breast implants with new implants that maintain breast projection and volume. According to these embodiments, breast implants for replacement of a previously implanted breast implant are provided. The combination of an internal skeleton element enclosed within a shell filled with a filler substance, and optionally additional reinforcing and anchoring structures, allows the breast implants disclosed herein to maintain the shape and contour of the breast achieved by the previous implant, with improved support and reduced weight.

The filler substance filling the breast implants provided herein may be a silicone gel or saline, or may be a non-liquid filler such as closed-cell silicone foam, foam beads or another substance as may be decided by a manufacturer. The filler substance may have specific density lower than 1.0 gr/cc. The implant may be filled with a filler having specific gravity lower than 0.5. According to some embodiments, the term “specific gravity” may refer to the ratio of the density of a substance (such as the implant or the implant filling) to the density of a reference substance (such as water). In some embodiments, compressible filler may be used, such as gas. According to these embodiments, the implant is partially compressible.

According to one aspect, there is provided a breast implant (a three dimensional breast implant) comprising: an internal skeleton element comprising a base configured to face a subject's chest wall when implanted; a dome configured to face a subject's nipple-areola complex when implanted; and an elongated projecting structure extending between said base and said dome; and a shell enclosing said internal skeleton element and filled with a filler substance, the implant is configured to be inserted into a sub-glandular plane, a sub-muscular plane or a subcutaneous plane of a breast to affect projection and volume of breast. In some embodiments, the filler substance is a liquid. In other embodiments, the filler substance is a non-liquid material. A non-liquid filler substance may be a closed cell silicone foam. The filler substance may have specific gravity lower than 0.5.

In some embodiments, the base has a first diameter and the dome has a second diameter, and the first diameter is larger than the second diameter.

In some embodiments, the internal skeleton element further comprises at least one reinforcing structure extending between the projecting structure and the dome, between the projecting structure and the base, or between the projecting structure, dome and base. The at least one reinforcing structure may be a planar structure. The planar structure may be a triangular structure (e.g. a sail-like structure). The at least one reinforcing structure may be a cord structure. The at least one reinforcing structure may be formed of or comprises a non-stretchable material.

In some embodiments, the shell comprises a protruding anchoring structure on an inner surface thereof and the dome comprises a matching indention on an outer surface thereof, and the protruding anchoring structure is configured to engage with the matching indention to hold the shell and the internal skeleton element in a fixed position with respect to each other.

In some embodiments, the dome comprises one or more radial ribs.

In some embodiments, the projecting structure comprises at least one pillar. In some embodiments, the projecting structure comprises at least one spring or spring-like structure.

In some embodiments, the base, the dome and the projecting structure are integrally formed. In other embodiments, at least one of the base, the dome and the projecting structure is formed as a separate component, which is configured to be assembled with remaining one or more components to form the implant.

In some embodiments, the implant is at least partially made of human biocompatible materials.

In some embodiments, the implant is at least partially coated with a human biocompatible coating. In some embodiments, the human biocompatible coating is a coating reducing tissue reaction to the implant. In some embodiments, such biocompatible coating is made of non-resorbable materials. In other embodiments, such biocompatible coating is made of resorbable materials and is gradually degraded when the implant is in the subject's body.

In some embodiments, the implant or at least parts thereof are formed of or comprise one or more resilient materials allowing natural movement of the breast.

In some embodiments, the implant is configured to be assembled during surgery.

In some embodiments, the implant is configured for defining the projection of the breast during surgery.

In some embodiments, the implant is configured for altering the projection of the breast post-surgery.

In some embodiments, an angle between the dome and the projecting structure can be any angle. In some embodiments, an angle between the base and the projecting structure can be any angle. Each of the angles (dome-projecting structure and base—projecting structure) may be independently designed and determined.

In some embodiments, the dome, the projecting structure and the base are manufactured from materials having different mechanical and/or chemical properties.

In some embodiments, an external surface of the implant is textured.

In some embodiments, at least one of the dome, the base, and the projecting structure comprises a body and an outer surface, the body and the outer surface are made from different materials.

In some embodiments, the projection is transiently changeable in response to pressure applied to it.

According to an additional aspect, there is provided a breast implant (a three dimensional implant) comprising: a skeleton element comprising a base configured to face a subject's chest wall when implanted; a dome configured to face a subject's nipple-areola complex when implanted; and a projecting structure having a lower portion and an upper portion and extending between said base and said dome; and a shell and a filler substance, wherein the lower portion of the projecting structure is enclosed within the shell, and wherein the upper portion of the projecting structure and the dome protrude outside the shell.

An implant according to embodiments of the present invention may further comprise a non-electromagnetic energy emitting and non-erasable code identifier on a surface thereof or embedded therein, for non-invasively identifying the implant when implanted in a subject. The code identifier may be embedded in, or attached to, the shell of the implant and made of a material having mechanical properties which are similar to the mechanical properties of the shell. The code identified may be partially embedded within the shell of the implant.

More details and features of the current invention and its embodiments may be found in the description and the attached drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below:

FIGS. 1A, 1B and 1C are illustrations of internal and outer subassemblies of a breast implant according to embodiments of the present invention. FIG. 1A is a sectional side view of an outer shell; FIG. 1B is a sectional side view of one embodiment of a bottom sealing patch and an internal skeleton; and FIG. 1C is a sectional side view of another embodiment of a bottom sealing patch and an internal skeleton.

FIG. 2 is an illustration of a fully assembled breast implant according to embodiments of the present invention comprising two subassemblies.

FIG. 3 is a schematic lateral section of a human breast with a breast implant according to embodiments of the present invention placed within the breast tissue.

FIG. 4 is a cross-section side view illustration of yet another embodiment of a breast implant, configured to provide increased breast parenchyma projection and support.

FIG. 5 is a schematic lateral section of a human breast with a breast implant providing additional intra-parenchymal projection to the breast.

### DETAILED DESCRIPTION OF THE INVENTION

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following embodiments and aspects thereof are described and illustrated in conjunction with systems, tools and methods which are meant to be exemplary and illustrative, not limiting in scope.

Breast implants are provided herein with improved support and relatively reduced weight. The implants according to embodiments of the present invention may be in a shape of a breast mound, an egg shape, a triangular three-dimensional shape or another shape.

The breast implants may be used as primary implants. The breast implants may be used as secondary, replacement implants, for replacing a previously implanted breast implant following extraction thereof, configured for at least retaining the breast's contour and volume.

The breast implants may also be used for reconstructing tissue defects, resulting for example from surgery, trauma or birth defects. The shape of the implant may be any shape needed to fill a gap in a tissue in order to restore or achieve a desired contour.

The breast implants can be placed under a breast glandular tissue, under chest muscles or under subcutaneous tissues or flap tissue.

The breast implants may be filled or fillable with a filler having specific gravity lower than 0.5. In some embodiments the filler of implants disclosed herein may have specific gravity lower than 0.4. In additional embodiments the filler may have specific gravity lower than 0.3.

In the description and claims of the application, each of the words “comprise” “include” and “have”, and forms thereof, are not necessarily limited to members in a list with which the words may be associated.

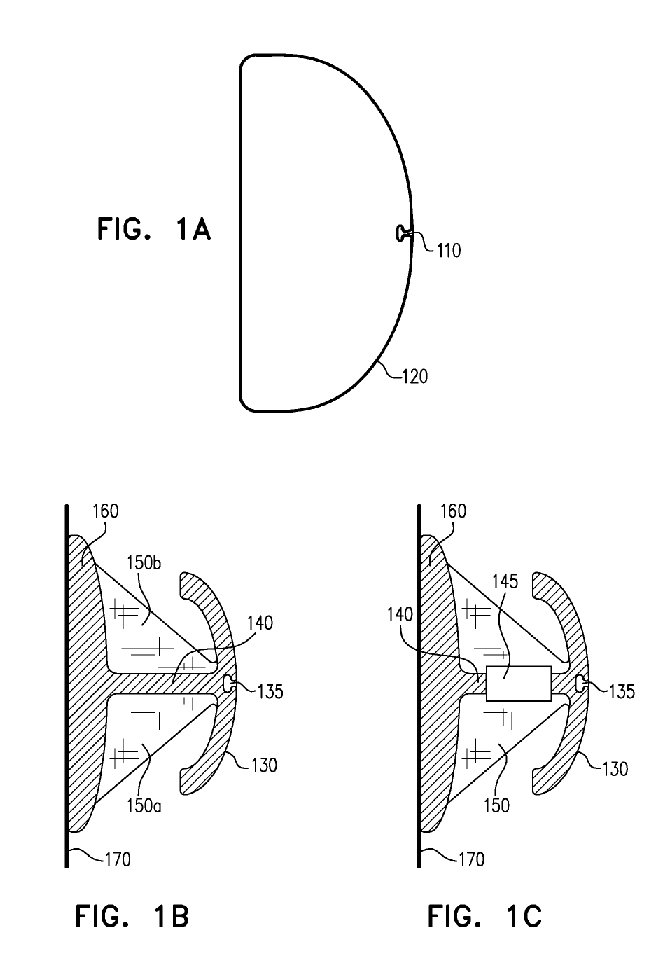

Reference is now made to FIG. 1A, FIG. 1B and FIG. 1C, which illustrate sectional side views of a top subassembly (FIG. 1A) and two possible supporting and sealing subassemblies (FIGS. 1B-1C) of a breast implant according to some embodiments of the present invention. The two subassemblies are configured to be assembled together to form a complete breast implant.

The top subassembly shown in FIG. 1A comprises a shell 120 and a protruding anchoring structure 110. In effect, FIG. 1A shows a first, top portion of shell 120, which is a convex structure open at its base to allow assembly with the supporting and sealing subassembly (FIGS. 1B-1C). In some embodiments, following assembly, the first portion of shell 120 is sealed to a bottom seal flap 170 (FIGS. 1B-1C), that constitutes a second portion of the shell. The first portion of shell 120 may be sealed to bottom seal flap 170, for example, using a glue, to generate a sealed shell enclosing the internal supporting structure.

In the illustrated embodiment, protruding anchoring structure 110 is at the apex of shell 120, protruding inwardly. Protruding anchoring structure 110 is configured to hold shell 120 and dome 130 (supporting and sealing subassembly in FIG. 1B-1C) in a fixed position with respect to each other, by engaging with a matching indention in the internal supporting structure.

The supporting and sealing subassembly shown in FIG. 1B comprises a central projecting structure 140 connecting dome 130 and base 160. Dome 130 is configured to face the nipple-areola complex of a subject when implanted, and the external surface of base 160 is configured to face, or be in contact with, the chest wall or tissue adjacent to the chest wall of the subject when implanted, thus supporting the implanted breast. As shown in FIG. 1B, projecting structure 140 is configured to affect the breast projection, depending on the length and angle thereof. An indention 135 at the tip of dome 130 is configured to accommodate protruding anchoring structure 110 of the top subassembly (FIG. 1A), thereby providing an attachment between dome 130 and the inner aspect of shell 120.

In the illustrated embodiment, projecting structure 140 is in the form of a pillar connected on one side thereof to a center of dome 130, and in an opposing side thereof to a center of base 160. In the illustrated embodiment, the diameter of dome 130 is smaller than the diameter of base 160. According to other embodiments, the diameter of the dome may be the same as the diameter of the base. Other proportions between the two diameters may be used, as desired by a manufacturer.

In the illustrated embodiment, base 160 is rounded and substantially flat. According to other embodiments, the base may be substantially flat with its edges being curved downwardly. According to further embodiments, the base may be convex, having smaller degree of convexity compared to the dome.

The width of the projecting structure is smaller than the diameters of the dome and the base. In some embodiments, the width of the projecting structure may range from about 5 mm to about 20 mm. In some embodiments, the diameter of the dome may range from about 20 mm to about 70 mm. In some embodiments, the diameter of the base may range from about 40 mm to about 120 mm.

In some embodiments, thickness of each of the dome and base may range from about 2 mm to about 7 mm. In some embodiments, the dome, the base or both are of a uniform thickness. In other embodiments, the dome, the base or both are of varying thickness. In some embodiments, the dome and the base are of the same thickness. In other embodiments, the dome and the base are of different thicknesses.

In some embodiments, the length of projecting structure may range from about 40 mm to about 150 mm.

According to some embodiments, dome 130 may be asymmetrical. Dome 130 is typically convex, but according to some embodiments it may be flat. According to some embodiments, dome 130 may be made of finger like projections rather than a full dome.

The aforementioned descriptions are only examples of options related to the relative sizes of dome 130 and base 160. Various shapes and sizes may be used for dome 130 and base 160 as desired and defined by a manufacturer, for different types of breasts to be implanted with implants.

In the illustrated embodiment, the supporting and sealing subassembly is further connected to, or integrally formed with, bottom seal 170 that together with the top portion of shell 120 forms a complete and sealed shell enclosing an inner volume (the inner volume of the implant), including the supporting structure. Bottom seal 170 is extended beyond base 160, namely the diameter of the bottom seal is greater than the diameter of the base. In the illustrated embodiment the supporting and sealing subassembly further comprises reinforcing structures 150 a,b. The reinforcing structure(s) according to embodiments of the present invention (which may include one or more such structures) may be have a sail-like shape (as shown in FIGS. 1A and 1B, 150 a,b) or may have any other form or shape such as, but not limited to, a cord, spring, string, planar structure, mesh, rib or any combination thereof. The reinforcing structure(s) according to embodiments of the present invention may be made of a resilient polymer and extend between the projecting structure and the dome, between the projecting structure and the base, or between the projecting structure, dome and base. In the illustrated embodiment, structures 150 a,b extend between projecting structure 140, base 160 and dome 130. The reinforcing structures 150 a,b are configured to limit bending of projecting structure 140 due to gravitational forces or other forces applied to the breast implant when implanted in the human body, and stabilize the projection and shape of the breast implant. For breast implants filled with a liquid filler, the reinforcing structures may also be configured to stabilize the shape of the breast implant by reducing movement of the liquid inside the implant. Such restriction of liquid movement also facilitates a more natural appearance and tactility of the implant.

The reinforcing structures may include a non-stretchable layer like a mesh as an example embedded therein to prevent stretching and further limit bending of the reinforcing structure due to gravitational forces or other forces applied to the breast implant when implanted in the human body. A “non-stretchable layer” refers to a layer that does not stretch (elongate) to more than about 10% relative to its initial dimensions upon application of stretching forces. Preferably, a non-stretchable layer does not stretch to more than about 1-5% relative to its initial dimensions upon application of stretching forces. The non-stretchable layer may be made of, or comprise, materials such as polyester, metal or polyamide woven, knitted or nonwoven fabrics.

A plurality of reinforcing structures may be molded between the projecting structure and the dome, between the projecting structure and the base, or between the projecting structure, dome and base, for example, two, three or four reinforcing structures. The reinforcing structures may be molded such that they oppose one another to limit movement of the projecting structure, thus stabilizing the overall shape of the implant when outside forces are applied to it. The number of reinforcing structures is defined during manufacturing according to design and engineering considerations. It is to be understood that the reinforcing structures are not limited to the triangular shape shown in FIG. 1B and may be shaped, for example, as strings, or other shapes as desired by a manufacturer. The reinforcing structures may have different dimensions or shapes as dictated by the mechanical properties needed according to their position in implant. The reinforcing structures (like the rest of the implant) may be made from a biocompatible material such as silicone or polyester.

In general, the projecting structure, dome and base are made of resilient materials allowing them to respond to pressure applied to the breast. The projecting structure and one or more reinforcing structures may be made of the same material and have substantially similar mechanical properties varying only due to differences in their structure and dimensions. Alternatively, the projecting structure and the reinforcing structures may be made of different materials, for example silicone polymers of different Shore A numbers, thus having different mechanical properties due to differences in pre-defined polymer qualities. For example, the projecting structure may be made of a silicone polymer having a lower Shore A number than the reinforcing structures, thus allowing the projecting structure to resiliently change its shape and length in response to pressure applied to it, in comparison to the reinforcing structures that are more rigid.

FIG. 1C shows another embodiment of a supporting and sealing subassembly. As in FIG. 1B, projecting structure 140 is configured to affect the breast projection, depending on the length and angle thereof. In this structure, however, projecting structure 140 and reinforcing structure 150 have a central void 145 configured to enable the projecting structure and reinforcing structure to partially collapse when pressure is applied to the breast. Once pressure is removed, projecting structure 140 and reinforcing structure 150 resume their original structure due to their resiliency. Thus, the void enables to transiently change a distance between the dome and the base in response to pressure applied to the breast. The ability to transiently change the distance between the dome 130 and base 160 in response to pressure applied to a breast implanted with a breast implant as described herein, confers the implant tactile feedback properties resembling human breast tissue.

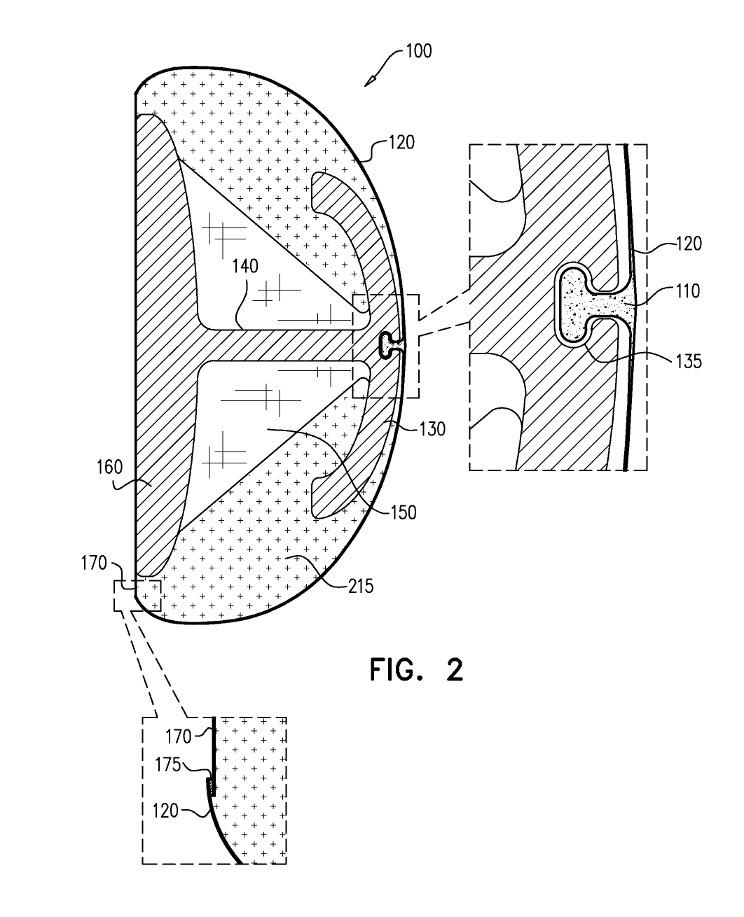

Reference is now made to FIG. 2, which shows a sectional side view of a fully assembled breast implant 100. Any of the parts constructing breast implant 100, namely, shell 120, dome 130, projecting structure 140 and base 160, may be manufactured as a separate part that is assembled with the rest of the parts to construct the complete breast implant 100. FIG. 2 further shows a close-up view of the anchoring point between dome 130 and shell 120, where protruding anchoring structure 110 is engaged within indention 135 to keep both parts together in a fixed position with respect to each other. Filler material 215 fills the vacant space inside shell 120. Filler material 215 may be a liquid, such as silicone gel or saline as non-limiting examples. Alternatively or additionally, filler material 215 may be a non-liquid, such as closed-cell silicone foam or any other material or a mix of materials suitable for long term implantation in human subjects and preferred by manufacturers. FIG. 2 further shows a close-up view of the point in which the top portion of shell 120 is sealed to bottom seal 170, using glue 175.

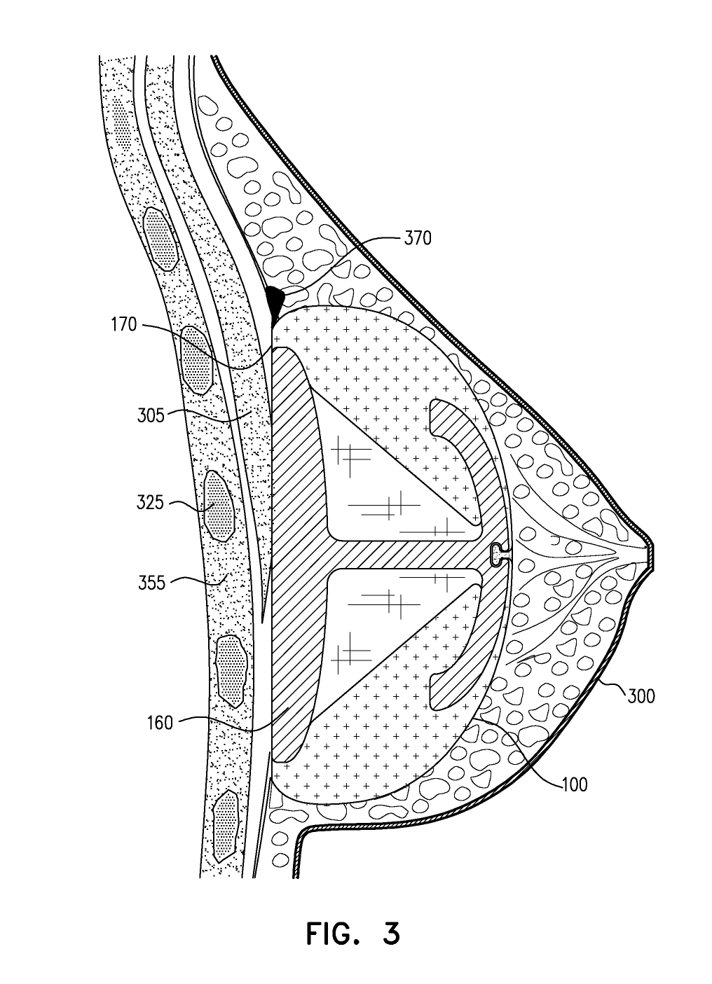

Reference is now made to FIG. 3, which shows breast implant 100 within breast 300 after implantation. Base 160 and bottom seal 170 lie against the chest wall comprising muscles 305, and against the rib cage comprising ribs 325 and intercostal muscles 355, supporting breast implant 100 in position and posture. Bottom seal 170 may have one or more base extensions 370 of various dimensions as designed by manufacturer extending beyond the outer diameter of breast implant 100. These base extensions 370 are placed in specially dissected sub-glandular pockets matching the size of base extensions 370 extending out of the pocket dissected to accommodate the implant 100 by the surgeon. In case of replacement of a previous implant, the pockets are dissected in addition to the capsule formed around the previous implant and defining the accommodating space for implant 100. The base extensions 370 are configured to prevent the circular movement of breast implant 100 in the pocket or capsule accommodating it and also prevent breast implant 100 from flipping over inside the pocket resulting in having base 160 facing close to the nipple-areola complex and the dome of the implant close to chest wall.

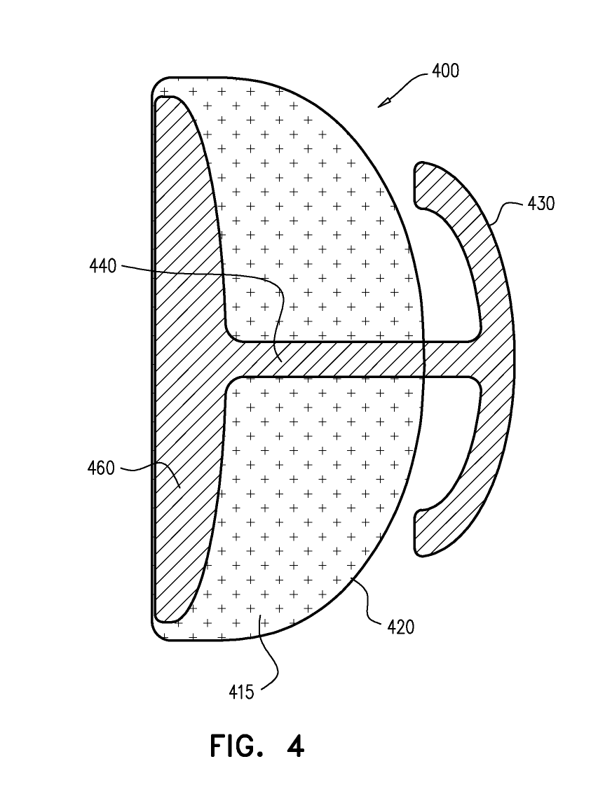

Reference is now made to FIG. 4, which shows a sectional side view of another design of a breast implant in accordance with some embodiments of the present invention. Breast implant 400 is configured to provide increased projection to the breast parenchyma. Breast implant 400 comprises a shell 420 filled with a filler substance 415, a base 460, a dome 430 and a central projecting structure 440 extending between base 460 and dome 430. Projecting structure 440 protrudes through shell 420 such that dome 430 is outside and above shell 420. Thus, a lower portion of the projecting structure and the base are enclosed within the shell, while an upper portion of the projecting structure and the dome protrude outside the shell and contact surrounding breast parenchyma when implanted. The dome, base and projecting structure may be integrally formed. Alternatively, at least one of the dome, base and projecting structure may be formed as a separate component configured to assemble to one or more remaining components to form the complete structure.

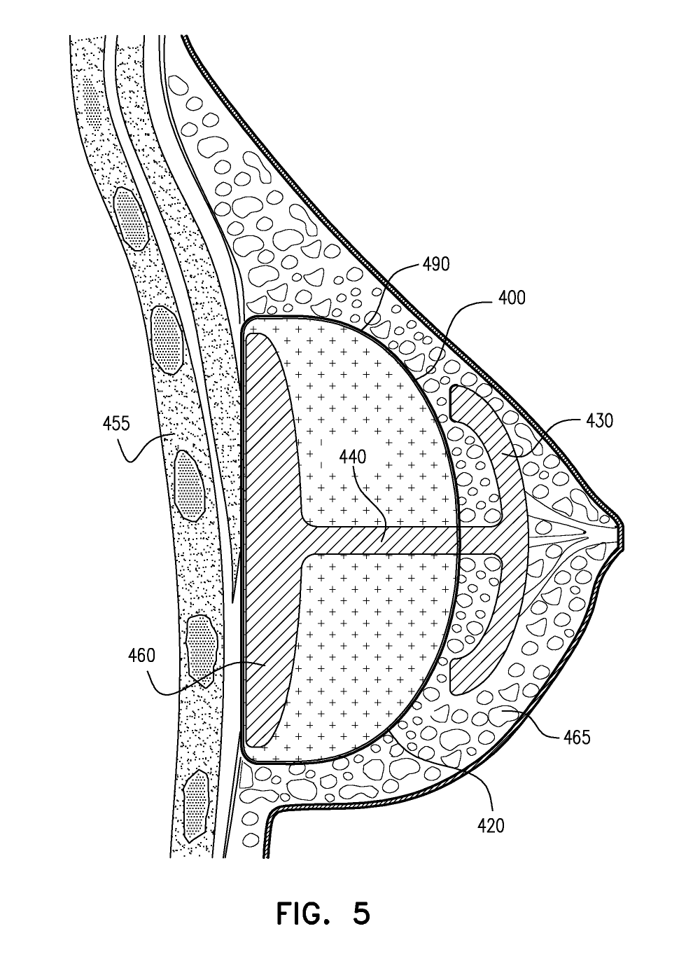

Reference is now made to FIG. 5, which shows breast implant 400 placed within a woman's breast. Breast implant 400 is surrounded by breast parenchyma 465, where shell 420 is in contact with the breast parenchyma and with a capsule 490 formed in the breast around the older implant. Base 460 is resting against chest wall 455. The portion of the implant comprising dome 430 and upper end of projecting structure 440 is placed within a specially dissected pocket in breast parenchyma 465, thus pushing breast parenchyma 465 upward and anteriorly.

An external surface of breast implants according to embodiments of the present invention may be smooth. Alternatively, breast implants according to embodiments of the present invention may include a textured external surface. An “external surface” is a surface configured to contact surrounding breast tissue when the implant is in the body. Breast implants according to embodiments of the present invention may be coated with a biocompatible material affecting tissue reaction to the implant, preferably reducing tissue reaction to the implant, such as reducing capsule contracture, like polyurethane or Parylene, as an example. Coating can be of the complete outer surface of the implant's shell or in patches covering only partially the outer surface of the breast implant. Capsule reducing properties can also be achieved by having protrusions extending from the external surface of the shell, thus defining an open pore texture where surface texture comprises shell polymer and coating polymer protrusions. In some embodiments, such biocompatible coating is made of a non-resorbable material and constitutes a permanent coating layer of the implant. In other embodiments, the biocompatible coating is made of a resorbable material that is gradually degraded when the implant is in the body, like a PLLA mesh, for example.

Breast implants according to embodiments of the present invention may include a non-electromagnetic energy emitting and non-erasable code identifier (label) on their surface or embedded therein, for non-invasively identifying the implants after they are implanted. According to one embodiment the code identifier may be printed during the manufacturing process. “Ink” materials may include, e.g., polymers or other biocompatible chemical compounds, which can be distinguished and detected by optical, electro-optical, electromagnetic, ultrasonic detection means, or other detection means known in the art. For example, a printing material may include a radio-opaque material like barium sulfate, may contain gas bubbles to be detected by ultrasound, may contain a color different from the color of the implant layer on which it is printed or in which it is embedded to be detected optically, may contain magnetic material defining a different magnetic imprint for each code, or any combination of the above, but not limited to the above. According to another embodiment, the code may be cut by laser or any mechanical cutting device or method from a sheath made of the above described “ink” materials. The code may be generated by a computer, either randomly or according to a pre-determined algorithm. The code may be a printable character or any drawing or shape. The code may be saved to a computerized database to be retrieved when needed through a local network or over the web. The code can advantageously be retrieved non-invasively from the implant while the implant is still implanted in the patient, without the need to extract the implant by surgery. Once the code is retrieved, some or all information regarding the implant, the patient and the operating physicians can be retrieved over the web or in any other method as allowed by authorities. In preferred embodiments, the code identifier is made of a material having mechanical properties similar to those of the shell it is embedded in or attached thereto, thus not affecting the mechanical properties of the shell. If the code identifier is embedded in, or attached to, an outer shell, the code identifier preferably has the same tactility of the shell, thus being impalpable to a subject touching the implant from outside the body. The code identifier may be placed within any compartment of the implant. The code identified may be partially embedded in any component of the implant and partially protruding from the component. The code identifier is typically made of a material having mechanical properties that do not damage the integrity of any component of implant.

1. A three dimensional breast implant comprising:
an internal skeleton element comprising a base configured to face a subject's chest wall when implanted; a dome configured to face a subject's nipple-areola complex when implanted; and an elongated projecting structure extending between said base and said dome; and
a shell enclosing said internal skeleton element and filled with a filler substance, said implant is configured to be inserted into a sub-glandular plane, a sub-muscular plane or a subcutaneous plane of a breast to affect projection and volume of breast.

2. The implant of claim 1, wherein said filler substance is a liquid.

3. The implant of claim 1, wherein said filler substance is a non-liquid material.

4. The implant of claim 3, wherein said filler substance is a closed cell silicone foam.

5. The implant of claim 1, wherein said filler substance has specific gravity lower than 0.5.

6. The implant of claim 1, wherein said base has a first diameter and said dome has a second diameter, and wherein said first diameter is larger than said second diameter.

7. The implant of claim 1, wherein said internal skeleton element further comprises at least one reinforcing structure extending between said projecting structure and said dome, between said projecting structure and said base, or between said projecting structure, dome and base.

8. The implant of claim 7, wherein said at least one reinforcing structure is a planar structure.

9. The implant of claim 8, wherein said planar structure is a triangular structure.

10. The implant of claim 7, wherein said at least one reinforcing structure is a cord structure.

11. The implant of claim 7, wherein said at least one reinforcing structure is formed of or comprises a non-stretchable material.

12. The implant of claim 1, wherein said shell comprises a protruding anchoring structure on an inner surface thereof, and said dome comprises a matching indention on an outer surface thereof, and wherein said protruding anchoring structure is configured to engage with said matching indention to hold said shell and said internal skeleton element in a fixed position with respect to each other.

13. The implant of claim 1, wherein said dome comprises one or more radial ribs.

14. The implant of claim 1, wherein said projecting structure comprises at least one pillar.

15. The implant of claim 1, wherein said projecting structure comprises at least one spring or spring-like structure.

16. The implant of claim 1, wherein said base, said dome and said projecting structure are integrally formed.

17. The implant of claim 1, wherein at least one of said base, said dome and said projecting structure is formed as a separate component, which is configured to be assembled with remaining one or more components to form said implant.

18. The implant of claim 1, wherein said implant is at least partially made of human biocompatible materials.

19. The implant of claim 1, wherein said implant is at least partially coated with a human biocompatible coating.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A three dimensional breast implant comprising:
a skeleton element comprising a base configured to face a subject's chest wall when implanted; a dome configured to face a subject's nipple-areola complex when implanted; and a projecting structure having a lower portion and an upper portion and extending between said base and said dome; and
a shell and a filler substance,
wherein said lower portion of the projecting structure is enclosed within said shell, and wherein said upper portion of the projecting structure and said dome protrude outside said shell.

33. (canceled)

34. (canceled)

35. (canceled)
